(19) United States
(12) Patent Application Publication
(10) Pub. No.: US 2004/0048829 A1
(43) Pub. Date: Mar. 11, 2004

(54) Molecular inclusion compounds

(76) Inventors: Stephan Hausmanns, Wiesbaden (DE); Thomas Kiy, Frankfurt am Main (DE); Dirk Fabritius, Mainz (DE); Ivan Tomka, Zurich (CH)

Correspondence Address: HESLIN ROTHENBERG FARLEY & MESITI PC, 5 COLUMBIA CIRCLE, ALBANY, NY, 12203, US

(21) Appl. No.: 10/448,245

(22) Filed: May 29, 2003

Related U.S. Application Data
(63) Continuation of application No. PCT/EP01/13971, filed on Nov. 29, 2001.

(30) Foreign Application Priority Data
Nov. 30, 2001 (DE) 100 59 726.2

(51) Int. Cl. A61K 31/715; C08B 37/00

(52) U.S. Cl. 514/54; 536/123.12

(57) ABSTRACT

Molecular inclusion compounds consisting of at least (a) one linear, water-soluble polysaccharide and (b) one or more fatty acid or fatty acid derivative are disclosed. The polysaccharide may be obtained biocatalytically. Methods for manufacturing the molecular inclusion compounds and the use of the molecular inclusion compounds in pharmaceutical preparations, as an ingredient of functional foodstuffs in cosmetic preparations, and as food additives and supplements are also disclosed.

### CROSS REFERENCE TO RELATED APPLICATIONS

This application is a continuation of PCT EP01/13971, which was filed Nov. 29, 2001, and designated the United States. PCT EP01/13971 was published in German as WO 02/43768 on Jun. 6, 2002. It claimed the priority of German application 100 59 726.2, which was filed Nov. 30, 2000.

### FIELD OF THE INVENTION

This invention concerns molecular inclusion compounds comprising two elements: (1) a linear, water-insoluble polysaccharide and (2) a fatty acid or fatty acid derivative. It also relates to procedures for the manufacture and the use of these compounds.

### BACKGROUND OF THE INVENTION

The manufacture of micro- or nanocapsules for enclosing or encapsulating substances or mixtures of substances is the subject of a number of studies. Accordingly, microcapsules are subdivided either into finely distributed dispersions, in which the encapsulated material is imbedded in a foam-like matrix (e.g. KR 94-9400419), or in structures, in which the material to be encapsulated is not penetrated by the capsule material, but is only surrounded by it (e.g. Arshady et al. 1991, Polymer Eng. Sci. 30 (15), 905-914 and 915-924).

In both cases these are undefined multi-molecular aggregates. In the case of the dispersions the encapsulated material forms part of the multi-molecular aggregates. Furthermore, it is known that starch ingredients such as amylose and amylopectin can be used to produce the aforementioned micro-capsules.

In these cases the primary purpose of the microcapsules is the protection of the encapsulated material against external influence (e.g. heat, UV light, oxidation) but they can contribute also to better processability of the material (e.g. flow characteristics, tackiness, and transformation of liquid products into solid ones). Another possible application of micro-capsules is to improve sensory properties in oral applications.

It is known that native starches can be used in the formation of micro-capsules (MC). In this instance, inter alia, starches having a high proportion of resistant starches (RS) can be used, which are fermentively broken down in the large intestine and not as usually the case by pancreatic amylase in the stomach and the small intestine.

In contrast, complex compounds similar to the iodine-starch complexes have been described, in which one or a plurality of iodine or fatty acid molecules are incorporated into a starch helix (see FIG. 1). Such complexes will be hereinafter referred to as molecular inclusion compounds. Helical iodine-starch complexes and their utilization in medicinal and pharmaceutical applications have been described by Gehnt and Eskin in U.S. Pat. No. 5,955,101 and U.S. Pat. No. 5,910,318.

WO 94/17676 discloses a composition comprised of hydrolyzed starch used as matrix for incorporated lipophilic compounds.

DE 44 11 414 proposes a combination of molecular inclusion and dispersion. It discloses a product for enteral supply of fatty acids, wherein the latter comprise at least 10% of the product. In this instance, the fatty acids are finely dispersed in a plasticized starch matrix, wherein a portion of the fatty acids are at least section-wise included in an amylose helix. However, it remains unclear how high the respective percentage of fatty acid molecules is included in the amylose helix.

Thus, molecular inclusion compounds are known, which are based on native, i.e. branched, water-soluble starch or its degradation products, and which, depending on the local specialist knowledge, could be incorporated with a maximum of 4.6% by weight of fatty acids (see also: loading) (compare with Example 4 and Krüger et al. Monatsschr. Brauwiss. (1984) 37(12) p. 505-512). Fanta et al. described a complexing of 4.6% by weight of myristic acid in an amylase-rich starch (Carbohydr. Polym. (1999) 38(1) p. 1-6). However, materials and methods allowing much higher amounts of fatty acids (higher loading) would be desirable.

### SUMMARY OF THE INVENTION

In one aspect, the invention relates to a molecular inclusion compound, characterized in that it consists of at least (a) one linear, water-soluble polysaccharide obtained biocatalytically and (b) one or more fatty acid(s) or fatty acid derivative(s).

In another aspect, the invention relates to a method for manufacturing the molecular inclusion compound characterized in that a linear, water insoluble polysaccharide obtained biocatalytically is homogenized in a mixture with a lipophilic compound and, if required, an unbound surplus of the lipophilic compound is removed by extraction.

In a third aspect, the invention relates to the use of the molecular inclusion compounds as an active substance carrier or an active substance in pharmaceutical preparations, as an ingredient of functional foodstuffs (“functional foods”), in cosmetic preparations, and as an additive to foods, as a food supplement as well as a symbiotic in combination with probiotic microorganisms.

### BRIEF DESCRIPTION OF THE DRAWINGS

FIG. 1 represents a diagrammatic representation of the molecular inclusion compound (A): process of binding with a fatty acid in the polysaccharide helix; (B) completely included fatty acid.

FIG. 2 represents x-ray spectra for poly-a-1,4-D-glucan with 35% glycerin (1) and in addition, 2.5% (2), 5% (3) and 10% (4) palmitic acid.

### DETAILED DESCRIPTION OF THE INVENTION

Molecular inclusion compounds are well-suited for use in pharmaceutical preparations, as functional foods, in cosmetic preparations and as food additives or food supplements, since the included compounds, for example, are well-protected against molecular effects such as enzymatic action, for example. The areas of application of such molecular inclusion compounds depend very essentially on the characteristics of the materials used for enclosing.

For certain applications of the molecular inclusion compounds according to this invention it is particularly desirable that the coating materials used are resistant to the action of α-amylase so that the molecular inclusion compounds according to the invention are digested only later in the large intestine. This would prevent the molecular inclusion compounds from being hydrolytically or enzymatically broken down at a higher rate than the rate of normal digestion. It would be furthermore desirable that the included lipophilic molecules inside the molecular inclusion compound be released only in the large intestine, so that they can be directly absorbed by cells in the wall of the large intestine without previously coming into contact with pancreatic enzymes and be consequently cleaved or modified to a substantial extent. Their bioavailability would thus be particularly advantageously increased.

An object of the present invention is, therefore, to provide materials and methods by means of which quantitatively substantially higher complexing of fatty acids in so-called molecular inclusion compounds is achieved. A further object of the present invention is to provide molecular inclusion compounds and methods for their manufacture, wherein the materials used for inclusion have novel properties, which would open up new areas of application for molecular inclusion compounds or new benefits in their utilization. A further object of the present invention is to provide improved molecular inclusion compounds and the method for manufacturing them, which, on the basis of the materials used, are utilizable as an ingredient of human medicinal or veterinary medicinal compositions, as food and feed components as well as for cosmetic applications.

Said objects are achieved by providing a molecular inclusion compound, characterized in that it is comprised of at least (a) one biocatalytically obtained, linear, water-soluble polysaccharide and (b) one or a plurality of fatty acid(s) or fatty acid derivative(s).

Other preferred embodiments and objects of the present patent application are described in the claims.

The inventors found surprisingly that using absolutely unbranched, water-insoluble polysaccharides, obtainable for example by biocatalyzed methods, molecular inclusion compounds can be obtained having appreciably higher loading than those with native starches.

An object of the present invention is therefore monomolecular inclusion compounds prepared from biocatalytically produced, water-insoluble polysaccharides and helically complexed lipophilic molecules, such as fatty acids or their esters, wherein the quantity of helical complexed lipophilic compounds is at least 5% by weight relative to the weight of polyglucan used. Preferably, however, the quantity of helical complexed lipophilic compound is at least 7% by weight relative to the polysaccharide used, particularly preferred 9% by weight, most preferably more than 10% relative to the polysaccharide used

Biocatalytically produced, linear, water-insoluble polysaccharides homogenized in a mixture with a lipophilic compound and processed to a homogeneous matrix are used for manufacturing the molecular inclusion compounds according to the invention. A residual unbound excess of the lipid may be subsequently removed by extraction.

If required, a plasticizer (softener) can be added. When this is done, homogenization can be carried out by extrusion. It is obvious to the specialist in the art that additional components can be added, such as flavor enhancers or ingredients affecting appearance or facilitate the processing in general.

Preferred softeners according to the invention are odorless, colorless, and resistant to light, cold or heat, non- or only minimally hygroscopic, water-stable, non-hazardous, difficult to ignite and having as low a volatility as possible, neutrally reactive, miscible with polymers and excipients and having good gelation properties.

In particular, they should be compatible with the ingredients used and have gelation capability and softening action. Examples of suitable softeners are water, polyalcohols such as ethylene glycol, glycerin, propanediol, erythritol, mannitol, sorbitol, polyvalent aliphatic carboxylic acids, such as maleic acid, adipic acid, succinic acid, polyvalent hydroxyaliphatic carboxylic acids, such as lactic acid, 2-hydroxybutyric acid, citric acid, malic acid, dimethylsulfoxide, urea or other solvents for starch.

The specialist in the art is familiar with the use of softeners. The softeners are preferably used in the proportion of 2 to 50% by weight relative to the polysaccharide component of the mixture according to the invention.

Fragrances, flavors, binders and the like can also be added if, for example, it is intended for cosmetic or pharmaceutical use or for use as a foodstuff or food ingredient.

The degree of loading of native starches is 2-3% by weight of palmitic acid that cannot be washed out using chloroform. Surprisingly, this proportion increases to 7.7% by weight in the case of the molecular inclusion compounds according to the invention by using biocatalytically obtained, linear water-insoluble 1,4-α-D-polyglucan as polysaccharide. The fatty acid is released from the molecular inclusion compound only after breakdown by the appropriate enzyme or chemical hydrolysis under suitable conditions and can then be re-isolated.

“Linear, water-insoluble polysaccharides” as used in the context of the present invention refers to polysaccharides constructed from monosaccharides, disaccharides or other monomeric components in such a manner that monosaccharides, disaccharides or other monomeric components are linked together always in the same manner. Each of the basic units or components so defined has exactly two links, each to another monomer. The exception is the two terminal units, the two basic units forming the beginning and the end of the polysaccharide. These basic units have only one link to a further monomer. In the case of three links to a basic unit (covalent bonds), one refers to a branch. Linear, water-insoluble polysaccharides as used in the context of the present invention have no or only minimal branching of the polysaccharide chain. If present, the degree of branching is so low (≦1%) that it cannot be detected using conventional analytical methods, such as ¹³C- or ¹H-NMR spectroscopy. Conversely, if branching can be detected, such that there is more than one triply linked monomer unit for every hundred doubly linked monomer units, the polysaccharide is not a “linear polysaccharide” as the term is intended.

In the context of the present invention, “water-insoluble polysaccharides” means compounds belonging as defined in the German pharmacopoeia (DAB-Deutsches Arzneibuch, Wissenschaftliche Verlagsgesselschaft mbH, Stuttgart, Govi-Verlag GmbH, Frankfurt, 9th Edition. 1987) corresponding to classes 4 through 7 in “low solubility”, “difficult to dissolve”, “very difficult to dissolve or “practically insoluble” compounds.

In the case of the polysaccharide used according to the invention this means that at least 98% of the quantity used, in particular at least 99.5% under normal conditions (T=25° C., ±20%, p=101325 Pascal±20%) is insoluble in water (corresponding to class 4 or 5). Preferred water-insoluble polysaccharides used according to the invention can therefore be classified as class 4 according to DAB; that is, a saturated solution of the polysaccharide at room temperature and normal atmospheric pressure comprises 30 to 100 parts by volume of solvent, that is, water, per part by weight of substance (1 g of substance in 30-100 ml of water). According to the invention more preferable water-insoluble polysaccharides can be classified as class 5 of the DAB; i.e. a saturated solution of polysaccharide at room temperature and normal atmospheric pressure contains 100 to 1000 parts by volume of solvent, that is, water, per part by weight of substance (1 g of substance in 100-1000 ml of water). According to the invention, even more preferred are the water insoluble polysaccharides that are attributable to class 6 of the DAB; that is, a saturated solution of the polysaccharide at room temperature and normal atmospheric pressure contains 1,000 to 10,000 parts by volume of solvent, that is, water, per part by weight of substance (1 g of substance in 1,000 to 10,000 ml of water). According to the invention, the most preferred water-insoluble polysaccharides are those belonging to class 7 of the DAB; that is, a saturated solution of polysaccharide at room temperature and normal atmospheric pressure contains 10,000 to 100,000 parts by volume of solvent, that is, water, per part by weight of substance (1 g of substance in 10,000 to 100,000 ml of water).

Difficult to dissolve or practically insoluble polysaccharide, in particular, very difficult to dissolve to practically insoluble polysaccharides are preferred for the present invention.

“Very low solubility” corresponding to class 6 can be illustrated by the following description of an experiment:

One gram polysaccharide of interest is heated in one liter of deionized water to 130° C. under a pressure of one bar. The solution obtained remains stable for a short period of several minutes. When the solution has cooled under normal conditions, the dissolved substance precipitates out. After cooling to room temperature and separation by centrifugation at least 66% of the amount used can be recovered, while taking into account the experimental losses.

The water-insoluble polysaccharide of choice is poly-α-1,4-D-glucan.

In the context of the present invention, linear, water-insoluble polysaccharides prepared by a biocatalytic (synonymous with biotransformation) or fermentation method are preferred.

Linear polysaccharides prepared by biocatalysis (synonymous with biotransformation) in the context of this invention means that the linear polysaccharide is produced by catalytic reaction of monomer basic units such as oligomeric saccharides like mono- and/or disaccharides, with the aid of a so-called biocatalyst, usually an enzyme. Biocatalysis can be carried out either using living growing cells, inactivated cells, immobilized cells or using enzymes isolated or made by genetic engineering methods in a single- or multiphase system.

Linear polysaccharide obtained by fermentation as used in the present invention means linear polysaccharides prepared by using fermentative processes with the help of naturally occurring organisms, such as fungi, algae or bacteria, or using non-naturally occurring organisms obtained drawing upon the methods of genetic engineering and generally defined as modified natural organisms such as fungi, algae, or bacteria or by the use and aid of fermentive methods.

Linear polysaccharides according to the invention can be, in addition to the preferred poly-α-1,4-D-glucan, other glucans or other linear polysaccharides such as pullulan, pectin, mannan or polyfructan.

Furthermore, linear polysaccharides for the production of the molecular inclusion compounds disclosed according to the invention can be obtained also from the reaction of other non-linear polysaccharides, in that non-linear polysaccharides containing branches are treated with enzymes so that the branches are cleaved, such that after cleaving, linear polysaccharides result. Examples of enzymes used for this purpose are the amylases, iso-amylases, gluconohydrolases or pullulanases. In any case, the polysaccharides according to the invention must be strictly linear.

In a particularly preferred embodiment of the invention the polysaccharide used is poly-α-1,4-D-glucan. Preferably the 1,4-α-D-polyglucan is obtained by a biocatalytic method (biotransformation) using polysaccharide synthases, starch synthases, glycosyl transferases, α-1,4-glucan transferases, glycogen synthases, amylosucrases or phosphorylases.

The molecular weights MW of the linear polysaccharides used according to the invention can vary within a wide range of 10³g/mol to 107 g/mol. For the preferred linear polysaccharide poly-α-1,4-D-glucan, molecular weights of 10⁴ g/mol to 105 g/mol, particularly 2×10⁴ g/mol to 5×10⁴ g/mol are preferred.

Polysaccharides resistant to α-amylase according to the invention can be characterized in that the poly-α-1,4-D-glucan is modified by methods known per se.

Thus, for example, the 1,4-α-D-polyglucans can be chemically modified by etherification or esterification at the 2-, 3- or 6-positions. The specialist in the art is extensively familiar with these chemical modifications; compare, for example, the following literature:

1. Functional properties of Food Components, 2nd edition, Y. Pomerantz, Academic Press (1991).

2. Textbook of Food Chemistry [“Lehrbuch der Lebensmittelchemie”], Belitz & Grosch, Springer Verlag (1992).

3. “Citrate Starch Possible Application as resistant Starch in Different Food Systems”, B. Wepner et al., European Air Concerted Action, Abstract: air3ct94-2203, Functional Properties of Non-digestible Carbohydrates, Pro Fibre-Tagung, Lisabon, February 1998, p. 59.

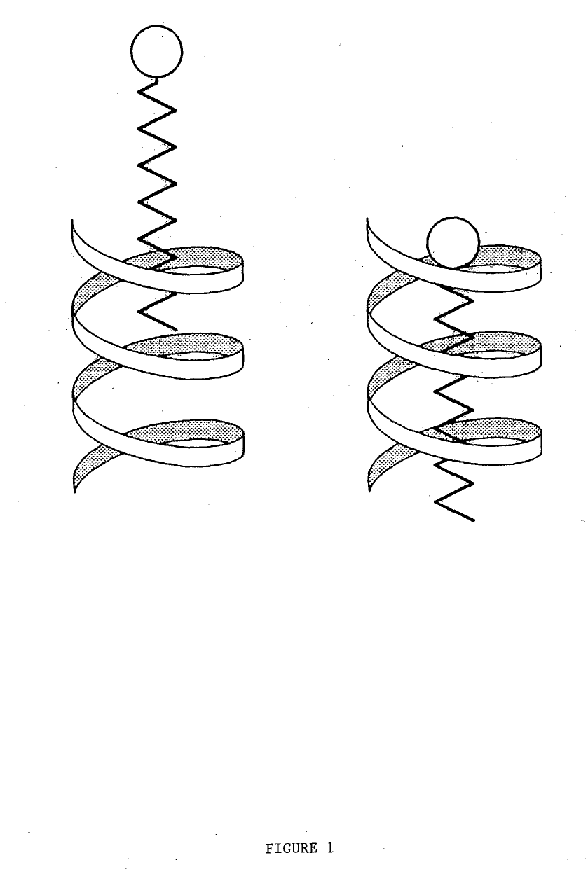

For the purposes of the present invention, the RS content is defined as the content of α-amylase resistant polysaccharides that can be determined by the method of Englyst et al. (Classification and measurement of nutritionally important starch fractions, European Journal of Clinical Nutrition, 46 (Suppl. 23) (1992) 33-50). For the purposes of the present invention the term “molecular inclusion compound” refers to complexes of lipophilic compounds with a polysaccharide (FIG. 1). As would be understood by the artisan, this means that the lipid is fixed by an interaction with the carbohydrate and not free. A molecular inclusion compound is thus conveniently quantified by determining the amount of lipid remaining in association with a carbohydrate after extraction with a solvent in which the lipid would dissolve, were it in the free state. Other means for quantifying the ratio of lipid to polysaccharide in a molecular inclusion compound include differential scanning calorimetry (DSC) and x-ray diffraction, discussed below.

The molecular inclusion compounds described in the present invention have, in a preferred exemplary embodiment of the present invention, in contrast with native starches, a high degree of resistance to α-amylase. In a particularly preferred exemplary embodiment of the present invention, the α-amylase-resistant inclusion compounds are characterized in that they have an RS-content according to Englyst of at least 30, preferably 50, particularly preferably 75, or most preferably 95% by weight. It is of great significance to the present invention that the biocatalytically obtained, linear and water-insoluble polysaccharides differ in a number of attributes from both native starches, as well as from the “enzymatically linearized” native starches disclosed in the prior art. The differences are summarized in Table 1, below.

TABLE 1
Differences between native starches biocatalytically obtained,
linear water-insoluble polysaccharides, particularly poly-α-
1,4-D-glucans, utilizable according to the invention.

| | Biotechnologically obtained, linear |
|---|---|
| | poly-α-1,4-D-glucans, usable |
| Native starch | according to the invention |
| Contains only polyglucans with | Strictly linear |
| branched chains | |
| Contains phosphoric acid esters of | Contains no esters of phosphoric |
| glucans | acid |
| Soluble in glycerin | Insoluble in glycerin |
| Crystalline portion corresponds | Can be produced as completely |
| maximally to the content of | crystalline by re-crystallization |
| amylopectin | |
| Cannot be precipitated from the | Can be precipitated from the solution |
| solution by crystallization | by crystallization |
| After melting the “A”-structure, it | “A”-structure can be re-stored by |
| cannot be restored by re-crystalli- | recrystallization |
| zation | |
| Film formation possible | Films cannot be formed |

Although they do not wish the invention to be held to a particular theory, the inventors hypothesize that the surprisingly higher loading capacity of the molecular inclusion compounds according to the present invention is not based on any single one of the characteristics of the material but rather that it is the sum of these features. It is possible that the strict linearity of the molecule according to the invention and the absence of phosphate esters combined with the low solubility in water in toto is responsible for the surprisingly advantageous properties of the inclusion compounds according to the invention.

Since the linear 1,4-α-D-polyglucan can be a matter of a more resistant form (RS>30%) compared to native starch, this has advantages in the oral administration of compounds, that must initially develop their action only after passing through the stomach and the small intestine.

Thus, for example, lipophilic active substances can be specifically released only in the large intestine.

Examples of the lipophilic substances that can be utilized according to the invention are the saturated or unsaturated fatty acids, the so-called PUFAs. As used for the purposes of the present invention PUFAs (polyunsaturated fatty acids) are defined as fatty acids with a chain length of more than 12 carbon atoms and with at least two double bonds (see table 2). Accordingly, the fatty acids can be used both in the form of free fatty acids, as esters of fatty acids, as physiologically compatible salts of fatty acids, as triglycerides or in the form of other derivatives.

The following Table represents a non-exhaustive compilation of particularly suitable fatty acids according to the invention.

TABLE 2
Particularly suitable fatty acids according to the invention.

| | IUPAC name | Trivial name | |
|---|---|---|---|
| C12 | Dodecanoic acid | Lauric acid | |
| C14 | Tetradecanoic acid | Myristic acid | |
| C14:1 | Cis-9-tetradecenoic acid | Myristoleic acid | |
| C15 | Pentadecanoic acid | | |
| C16 | Hexadecanoic acid | Palmitic acid | |
| C16:1 | Cis-9-hexadecenoic acid | Palmitoleic acid | |
| C16:1 | Trans-9-hexadecenoic acid | Palmitelaic acid | |
| C17 | Heptadecanoic acid | Margaric acid/ | |
| | | Standard | |
| C18 | Octadecanoic acid | Stearic acid | |
| C18:1 | Cis-9-octadecenoic acid | Oleic acid | |
| C18:1 | Trans-9-octadecenoic acid | Elaidic acid | |
| C18:1 | Trans(cis)-11-octadecenoic acid | | |
| C18:1 | Cis-6-octadecanoic acid | Petroselinic acid | |
| C18:2 | Cis,cis-9,12-octadecadienoic acid | Linoleic acid | ω-6 |
| C18:2 | Trans,trans-9,12-octadecadienoic acid | Linoleadic acid | ω-6 |
| C18:2 | Octadecadienoic acid, conjugated | Conjuenic acid | |
| C18:3 | All-cis-9,12,15-octadecatrienoic acid | α-Linolenic acid | ω-3 |
| C18:3 | All-cis-6,9,12-octadecatrienoic acid | γ-Linolenic acid, | ω-6 |
| | | GLA | |
| C18:4 | All-cis-6,9,12,15 -octadecatetraenoic | Stearidonic acid | ω-3 |
| | acid | | |
| C20 | Eicosanoic acid | Arachidic acid | |
| C20:1 | Cis-11-cicosenoic acid | | |
| C20:2 | Cis,cis-11,14-eicosadienoic acid | | ω-6 |
| C20:3 | All-cis-8,11,14-eicosatrienoic acid | Homo-γ-linolenic | ω-6 |
| | | acid | |
| C20:3 | All-cis-11,14,17-eicosatrienoic acid | | ω-3 |
| C20:4 | All-cis-5,8,11,14-eicosatetraenoic | Arachidonic acid, | ω-6 |
| | acid | ARA | |
| C20:4 | All-cis-8,11,14,17-eicosatetraenoic | ETA | ω-3 |
| | acid | | |
| C20:5 | All-cis-5,8,11,14,17-eicosapentaenoic | EPA, timnodonic | ω-3 |
| | acid | acid | |
| C22 | Docosanoic acid | Behenic acid | |
| C22:1 | Cis-13-docosenoic acid | Erucic acid | |
| C22:1 | Trans-13-docosenoic acid | | |
| C22:2 | Cis,sic-13,16-docosadienoic acid | | ω-6 |
| C22:3 | All-cis-13,16,19-docosatrienoic acid | | ω-3 |
| C22:4 | All-cis-7,10,13,16-docosatetraenoic | | ω-6 |
| | acid | | |
| C22:5 | All-cis-7,10,13,16,19- | DPA fish oil | |
| | docosapentaenoic acid | | |
| C22:5 | All-cis-4,7,10,13,16- | DPA Protisten | ω-6 |
| | docosapentaenoic acid | | |
| C22:6 | All-cis-4,7,10,13,16,19- | DHA | ω-3 |
| | docosahexaenoic acid | | |
| C24 | Tetracosanoic acid | | |
| C24:1 | Cis-15-tetracosenoic acid | Nervonic acid | |
| C26 | Hexacosanoic acid | | |

By inclusion in the particularly preferred α-amylase resistant molecular inclusion compounds according to the invention, these fatty acids can be protected against premature breakdown in the digestive system.

It is not necessary that the polysaccharide component of the compound according to the invention be a single polysaccharide; it can be a mixture of different water-insoluble, linear polysaccharides.

Applications for molecular inclusion compounds can be found inter alia in the disease areas of enteric cancer (Bougnoux 1999, Curr. Opin. Clin. Nutr. Metab. Care, 2 (2), 121-126; Wisont 1999, Inform 10 (5), 380-397), inflammatory enteric disorders (for example, Crohn's disease or colitis), mental or neurological disorders (for example, depression, schizophrenia, Alzheimer's) and vascular diseases (for example, high blood pressure, atherosclerosis).

Other important areas of application of the molecular inclusion compounds according to the invention are in the areas of pediatric nutrition, infant and newborn nutrition, clinical nutrition, functional foods, cosmetic applications and food additives.

The following examples explain the invention more completely.

### Example 1

Production of poly-α-1,4-D-glucan

5 L of a sterilized 30% saccharose solution is filled into a 5 L container. An enzyme extract, containing an amylosucrase from Neisseria polysaccharea (see WO 95 31 553) is added to a portion and mixed. The enzyme activity used in this experiment is 148,000 units. The closed container was incubated at 27° C. During the period of biotransformation a white precipitate forms. The reaction is terminated after 39 hours. The precipitate is separated by centrifugation, frozen at −70° C. and then freeze dried. The weight of the freeze-dried solid is 526.7 g (70.2% yield).

For separation of low molecular weight sugar, 200 g of the solid is washed with water for 30 minutes under agitation at room temperature, frozen at −70° C. and freeze dried. The fructose and saccharose content is determined after dissolving the solid in DMSO by means of a coupled enzymatic assay 1 and gives 4.61 mg fructose per 100 mg of solid (4.6%). The saccharose content is below the limit of detection.

The supernatant of the biotransformation is denatured at 95° C. After cooling to room temperature it is again centrifuged. The clear supernatant was frozen at −70° C. and thawed at 4° C. over 3 days. The precipitate produced in this manner was frozen at −70° C. and freeze dried.

For separating the low molecular weight sugar, 39.5 g of the solid was washed in water for 30 minutes under agitation at room temperature, frozen at −70° C. and freeze dried. The fructose and saccharose content is determined after dissolving the solid in DMSO by means of a coupled enzymatic assay according to Stitt et al. (Meth. Enzym., 174 (1989) 518-552) and is 2.27 mg fructose per 100 mg solid. The saccharose content is below the limit of detection.

### Example 2

Characterization of the Starting Material

Determination of the molecular weight of the synthesized water-insoluble poly-α-1,4-D-glucan from Example 1 (FIG. 1).

2 mg of the poly-α-1,4-D-glucan of Example 1 is dissolved at room temperature in dimethylsulfoxide (DMSO, p.a., Riedel-de-Haen) and filtered (2 (m). A part of the solution is infiltrated in a column for gel permeation chromatography. DMSO is used as the eluent. The signal intensity is measured using an RI detector and evaluated against a pullulan standard (Polymer Standard Systems). The flow rate is 1.0 ml per minute.

The measurement gives a numeric mean of molecular weights (Mn) of 1.326 g/mol and an average weight of molecular weights (Mw) of 3.367 g/mol. The recovery rate is 100%.

### Example 3

Determination of the maximal possible degree of complexing.

A mixture of 200 g poly-a-1,4-D-glucan (material from Example 1), 70 g of glycerin and 5 g, 10 g, 20 g, or 30 g of palmitic acid (corresponds to 2.5%, 5%, 10% or 15% relative to the weight proportion of the polyglucan) are prepared and homogenized in an extruder at 170° C. and 100 r.p.m. After cooling, samples are taken of the product. Using the DSC (digital scanning calorimetry) the melting peaks of the samples are determined. Then with the aid of a Soxhlet extraction (Chloroform, 48 h) by liberating the non-complexed palmitic acid, the degree of complexing or Kx is determined.

As the result of complex formation, palmitic acid is included in the amylose-helix; there is no longer a coherent palmitic phase. Only with a surplus of palmitic acid is the non-complexed part of the acid present as a coherent phase with its own melting peak. The absence of the palmitic acid melting peak is thus evidence that complexing has occurred completely. If there is a residual melting peak present, then the portion of complexed acid can be calculated from the difference in areas. These values are listed in Table 3 and compared to the results of the Soxhlet extraction. In the last column of Table 3 the further results of a Soxhlet extraction are given, wherein the production of the samples according to Example 1 native starch (purified potato starch) was used in lieu of poly-α-1,4-D-glucan.

TABLE 3
Results of DSC and Soxhlet Extraction

| | | | | Soxhlet- | Soxhlet- |
|---|---|---|---|---|---|
| | | Peak | Peak | Extract | Extract |
| Palmitic | Kx [%] | [° C.] | [° C.] | Linear | Native |
| Acid [%} | Heating | Heating | Cooling | Polyglucan [%] | Starch [%] |
| 2.5 | 2.5 | 71 | — | 2.2 | 1.9 |
| 5 | 5.0 | 65 | — | 4.7 | 2.4 |
| 10 | 7.6 | 65 | 34 | 7.4 | 2.5 |
| 15 | 7.7 | 66 | 49 | 7.5 | 2.5 |
| 100 | — | 68 | 49 | — | — |

The results allow the conclusion that, employing 35% glycerin as the softener, 7.5-7.7% palmitic acid, relative to the weight of the pure poly-α-1,4-D-glucan, can be complexed. On the other hand by using native potato starch a maximum complexing of 2.5% palmitic acid is possible.

### Example 4

Structural Study by X-Ray Diffraction.

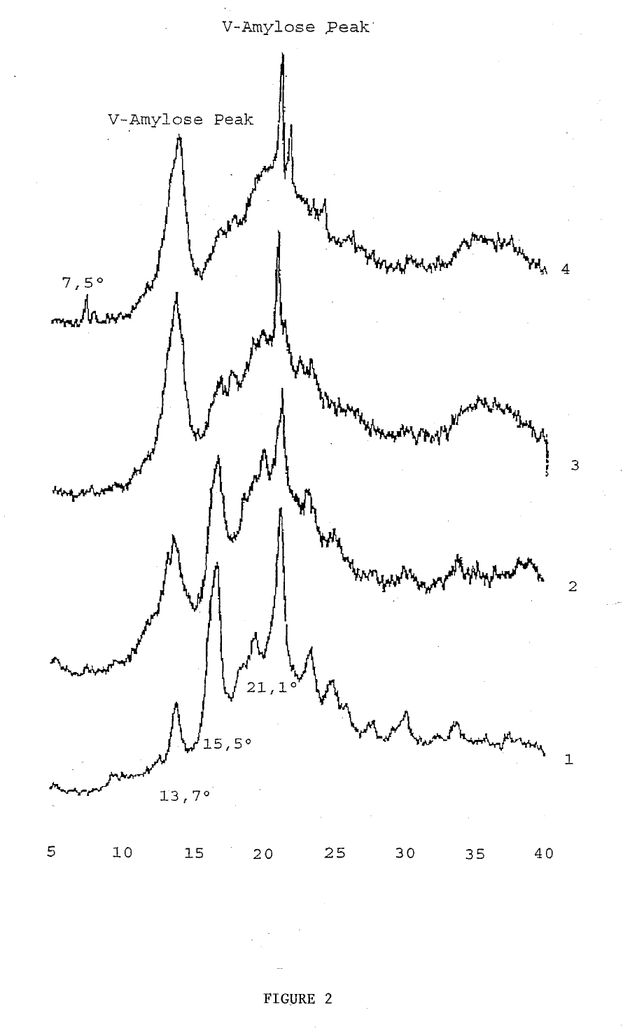

The samples described in Example 3 were subjected to an x-ray structural analysis. X-ray spectra for poly-α-1,4-D-glucan with 35% glycerin (1) and additional 1.5% (2), 5% (3) and 10% (4) palmitic acid are shown in FIG. 2. It can be seen that the spectrum for the pure, plasticized poly-α-1,4-D-glucan (1) from the amorphous halo, makes up the three major peaks at 13.7, 15.5 and 21.1° 22, and several smaller peaks. The peaks at 13.7 and 21.1° are characteristic for the simple helix of V-amylose, a structure type that is typical for complexed starches. In addition several other structures are present as evidenced by the peak at 15.5° and the various smaller peaks. These structures diminish with increasing palmitic acid content, while the peak associated with the V-amylose structure type continues clearly visible at 13.7°. This is only weakly depicted without a palmitic acid content and with increasing palmitic acid content ((2)→(4)) becomes more pronounced. With a maximal degree of complexing the precise crystalline phase in the V-amylose structure type is present. At 10% palmitic acid (4) a peak is observed at 7.5°, which can be attributed to the pure palmitic acid. The presence of non-complexed palmitic acid at this concentration is in accord with the results of the DSC measurement and the Soxhlet extraction. In the x-ray spectra the peak at 7.5° is only observed at 10% palmitic acid; at 2.5 and 5% no peak is observed.

1. A molecular inclusion compound comprising: (a) a linear polysaccharide having a water solubility of less than 1 g in 30 mL and (b) at least one fatty acid or fatty acid derivative.

2. The molecular inclusion compound according to claim 1 wherein the fatty acid or fatty acid derivative constitutes at least 5% by weight of the linear polysaccharide.

3. The molecular inclusion compound according to claim 2 wherein the fatty acid or fatty acid derivative constitutes at least 7% by weight of the linear polysaccharide.

4. The molecular inclusion compound according to claim 2 wherein the fatty acid or fatty acid derivative constitutes at least 9% by weight of the linear polysaccharide.

5. The molecular inclusion compound according to claim 2 wherein the fatty acid or fatty acid derivative constitutes at least 10% by weight of the linear polysaccharide.

6. The molecular inclusion compound according to claim 1 wherein the fatty acid or fatty acid derivative is chosen from the group consisting of free fatty acids, fatty acid esters, physiologically compatible salts of fatty acids and triglycerides.

7. The molecular inclusion compound according to claim 1 wherein the fatty acid or the fatty acid derivative has a chain length of from 12 to 30 carbon atoms and contains at least two carbon-carbon double bonds.

8. The molecular inclusion compound according to claim 1 wherein the polysaccharide is linear poly-α-1,4-D-glucan.

9. The molecular inclusion compound according to claim 1 wherein the inclusion compounds have a resistant starch (RS) content of at least 30% by weight of total polysaccharide as measured in an Englyst test.

10. The molecular inclusion compound according to claim 1 wherein the inclusion compounds have a resistant starch (RS) content of at least 50% by weight of total polysaccharide as measured in an Englyst test.

11. The molecular inclusion compound according to claim 1 wherein the inclusion compounds have a resistant starch (RS) content of at least 75% by weight of total polysaccharide as measured in an Englyst test.

12. The molecular inclusion compound according to claim 1 wherein the inclusion compounds have a resistant starch (RS) content of at least 95% by weight of total polysaccharide as measured in an Englyst test.

13. The molecular inclusion compound according to claim 1 wherein the linear polysaccharide has a water solubility of less than 1 g per liter.

14. The molecular inclusion compound according to claim 1 wherein the linear polysaccharide has a water solubility of 0.1 to 0.01 g per liter.

15. The molecular inclusion compound according to claim 1 wherein the linear polysaccharide is obtained biocatalytically.

16. A method for manufacturing the molecular inclusion compound of claim 1 comprising homogenizing said water insoluble polysaccharide with said at least one fatty acid or fatty acid derivative.

17. The method according to claim 16 additionally comprising the step of extracting unbound surplus of the fatty acid or fatty acid derivative.

18. The method according to claim 17 additionally comprising the step of adding a plasticizer to the mixture prior to homogenization.

19. A pharmaceutical preparation, functional foodstuff, cosmetic preparation, food additive or food supplement comprising the inclusion compound of claim 1.

20. An edible material comprising the inclusion compound of claim 1 and a probiotic microorganism.
